(19) United States
(12) Patent Application Publication
(10) Pub. No.: US 2023/0094714 A1
(43) Pub. Date: Mar. 30, 2023

(54) TRAY AND METHODS FOR MEDICAL PRODUCTS PACKAGING, STORAGE AND ACCESS

(71) Applicant: Potrero Medical, Inc., Hayward, CA (US)

(72) Inventors: Sanjay BANERJEE, Dublin, CA (US); Hannah JACKSON, San Diego, CA (US); Rich KEENAN, Granite Bay, CA (US); Dimitri SOKOLOV, Castro Valley, CA (US); Saheel SUTARIA, El Cerrito, CA (US)

(73) Assignee: Potrero Medical, Inc., Hayward, CA (US)

(21) Appl. No.: 17/930,504

(22) Filed: Sep. 8, 2022

Related U.S. Application Data
(63) Continuation of application No. PCT/US2021/001876, filed on Feb. 19, 2021.
(60) Provisional application No. 62/988,024, filed on Mar. 11, 2020.

(51) Int. Cl. A61B 50/30; A61M 25/00; A61M 5/00; A61B 42/40

(52) U.S. Cl. CPC A61B 50/30; A61M 25/002; A61M 5/002; A61B 42/40; A61B 2050/3008

(57) ABSTRACT

Trays and methods for medical products packaging, storage and access are described where a multi-level packaging assembly may comprise a top tray level, a lower tray level upon which the top tray level is removably positioned, one or more compartment barriers which define a first compartment within the lower tray level such that the first compartment is sized to accommodate a single syringe. The first compartment barriers may have a continuous ledge at a constant level with respect to a bottom of the lower tray level, and a base which is uninclined relative to the bottom of the lower tray level, and at least one layer of wrap sized so as to fold over and enclose both the lower tray level and the top tray level such that unfolding of the at least one layer of wrap reveals the top tray level.

### CROSS-REFERENCE TO RELATED APPLICATIONS

This application is a continuation of International App. No. PCT/US2021/018760 filed Feb. 19, 2021, which claims priority to U.S. Provisional Application No. 62/988,024 filed Mar. 11, 2020, each of which is herein incorporated by reference to the same extent as if each such individual publication or patent application were specifically and individually indicated to be so incorporated by reference.

### FIELD

Embodiments disclosed herein relate to devices and methods for packaging and accessing medical devices and accessories.

### INCORPORATION BY REFERENCE

All publications and patent applications mentioned in this specification are herein incorporated by reference to the same extent as if each such individual publication or patent application were specifically and individually indicated to be so incorporated by reference.

### BACKGROUND

Medical devices, and particularly disposable medical devices, supplies, accessories, etc., are generally shipped and stored in sterile packaging.

While conventional packaging works well for objects having a generally unchanging form factor, special considerations have to be taken into consideration for some medical supplies. By way of example, catheter assemblies may come packaged with several accessories. The components and/or accessories may be utilized in particular sequences. For example, it may be desired to access the sterile gloves first, so that they may be donned first before touching other contents of the packaging.

### SUMMARY

Embodiments of packaging systems disclosed herein allow for different components of the packaging to be accessed as needed. For example, components, such as sterile gloves, may be placed on a top tray level so that they may be accessed before revealing the contents of the lower tray level. This may serve to prevent contamination of the sterile contents of the lower tray.

One embodiment of a multi-level packaging assembly may generally comprise a top tray level, a lower tray level upon which the top tray level is removably positioned, one or more compartment barriers which define a first compartment within the lower tray level such that the first compartment is sized to accommodate a single syringe, wherein the first compartment barriers have a continuous ledge at a constant level with respect to a bottom of the lower tray level, a base defined within the first compartment, wherein the base is uninclined relative to the bottom of the lower tray level, and at least one layer of wrap sized so as to fold over and enclose both the lower tray level and the top tray level when the top tray level is positioned upon the lower tray level such that unfolding of the at least one layer of wrap reveals the top tray level.

One method of using a multi-level packaging assembly may generally comprise unfolding at least one layer of wrap enclosing a top tray level positioned upon a lower tray level to reveal the top tray level, and removing the top tray level from the lower tray level to access the lower tray level. The lower tray level may comprise one or more compartment barriers having a continuous ledge at a constant level with respect to a bottom of the lower tray level and which define a first compartment having a single syringe positioned within the first compartment, and the first compartment may further defines a base which is uninclined relative to the bottom of the lower tray level.

One method of producing a multi-level packaging assembly may generally comprise providing a lower tray level having one or more compartment barriers and a base which together define a first compartment within the lower tray level, wherein the one or more compartment barriers have a continuous ledge at a constant level with respect to a bottom of the lower tray level, and wherein the base is uninclined relative to the bottom of the lower tray level, securing a single syringe within the first compartment and one or more instruments within the lower tray level, positioning a removable top tray level upon the lower tray level, and enclosing the lower tray level and the top tray level with at least one layer of wrap such that unfolding of the at least one layer of wrap reveals the top tray level.

### BRIEF DESCRIPTION OF THE DRAWINGS

FIG. 1 shows an embodiment of the sterile catheter and catheter accessory packaging.

FIG. 2 shows the embodiment shown in FIG. 1 as it is being unwrapped.

FIG. 3 shows the embodiment shown in FIG. 1 after the sterile wrap has been unwrapped to reveal the packaging tray.

FIG. 4 shows the embodiment shown in FIG. 1 after the gloves and drape have been removed from the top tray level.

FIG. 5 shows the embodiment shown in FIG. 1 as the top tray level is being removed from the lower tray level.

FIG. 6 shows the tray shown in FIG. 5 after the top level tray has been removed.

FIGS. 7 and 8 show an angled view of the tray shown in the above figures.

FIG. 9 shows the lower tray without its contents.

FIGS. 10 and 11 show another embodiment of the tray packaging system.

FIG. 12 shows the embodiment shown in FIGS. 10 and 11 as the upper tray level is being removed.

FIG. 13 shows an embodiment with some of the contents of the lower tray in their compartments.

FIG. 14 shows the current embodiment with some of the contents removed to reveal the various compartments and barriers.

FIG. 15 shows the bottom of tray 502 shown in FIGS. 10-14.

FIG. 16 shows some of the steps that may be used when using the disclosed packaging tray system.

### DETAILED DESCRIPTION

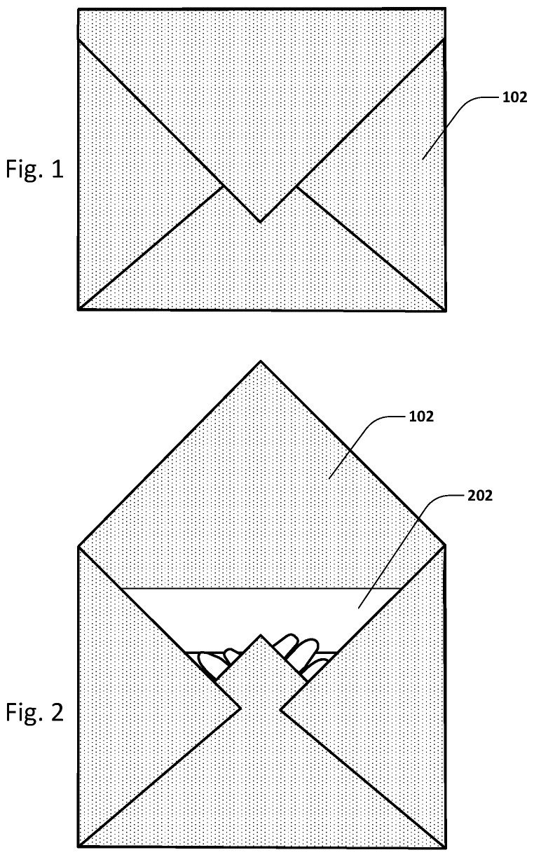

FIG. 1 shows an embodiment of the sterile catheter and catheter accessory packaging. FIG. 1 shows a double level tray wrapped in one or more pieces of sterile wrap 102. For example, the sterile wrap may be a paper composite wrap. The wrap may be square, rectangular, circular or any other shape. The wrap may be dimensioned so that it adequately covers the underlying tray, without adding excessive bulk. This sterile wrap may serve as both a sterile field and also to keep the layers of the tray together, and to help prevent shifting of the contents of the upper tray, before use.

The wrapped tray (packaging tray with the wrap wrapped around it, as shown in FIG. 1, may additionally be contained in a sealed sterile package, such as a sealed polymer package or bag. The sealing may be a thermal sealing.

FIG. 2 shows the embodiment shown in FIG. 1 as it is being unwrapped. One corner of sterile wrap 102 has been unfolded to reveal top layer 202 of a double layer packaging tray. Other items in the packaging may also be revealed as the sterile wrap is unwrapped. In some embodiments, the folds, or corners, of wrap 102 are free of any items, such as hand sanitizer, additional wrap, instructions, etc.

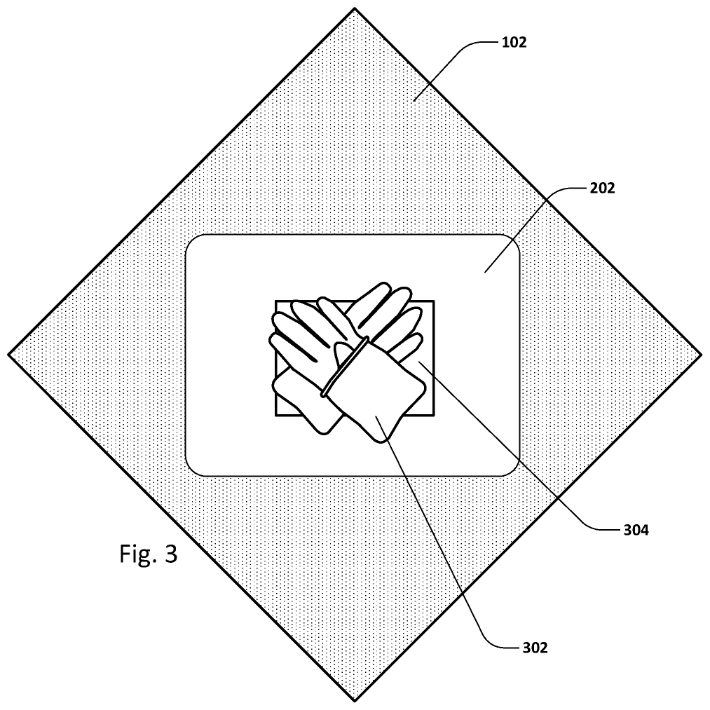

FIG. 3 shows the embodiment shown in FIG. 1 after sterile wrap 102 has been unwrapped to reveal the packaging tray. Wrap 102 also may act as a sterile field for unpacking the tray and for placing other items. Shown here is top tray level 202, sterile gloves 302 and sterile drape 304. Gloves 302 and drape 304 are shown here on top of the top tray level. Items placed on the top level of the tray may be accessed more easily without risk of contaminating other items in the tray, especially items in the lower level(s) of the tray. For example, sterile gloves may be placed on the top tray level so that the user may access them, and put them on, before touching anything else in the packaging and risking contamination. In some embodiments, the gloves are revealed after sterile wrap 102 has been partially or fully unwrapped. Drape 304 may be used to cover a portion of the patient before, during and/or after the procedure, for example a fenestrated drape. Drape 304 and/or gloves 302 may be on the top tray level, or at lower tray levels, but preferably, those items that are needed earlier, are on the top tray level.

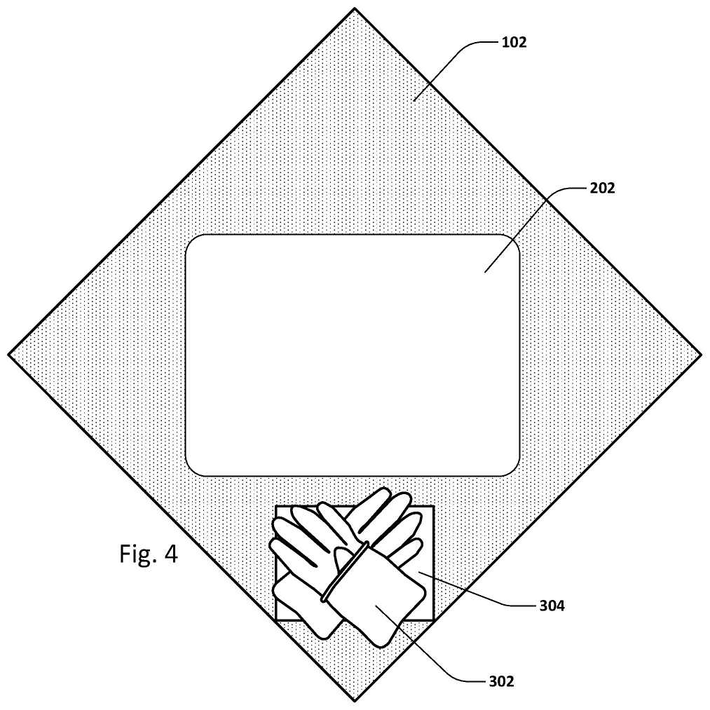

FIG. 4 shows the embodiment shown in FIG. 1 after gloves 302 and drape 304 have been removed from top tray level 202 and placed on sterile wrap 202. The gloves may alternatively be donned before the rest of the tray is accessed.

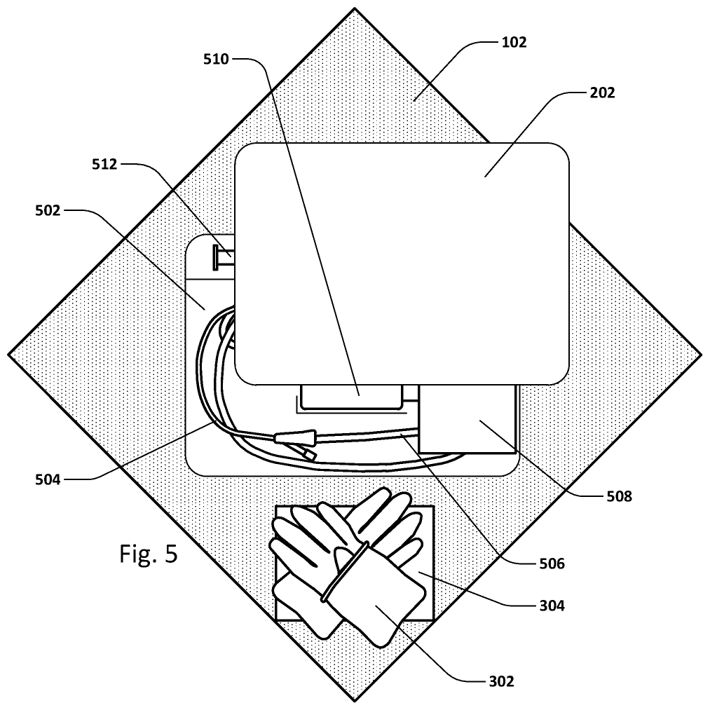

FIG. 5 shows the embodiment shown in FIG. 1 as top tray level 202 is being removed from lower tray level 502. Lower tray level 502 contains other items including in this example, catheter 504, which is connected to drainage line 506, which is connected to fluid receptacle 510, which is connected to drainage bag 508. In some embodiments not all of these items are present. For example, catheter 504 may not be present, or receptacle 510 may not be present. Also shown here is syringe 512, which may contain fluid for inflating a balloon catheter or may contain lubricant for aiding catheter insertion into the patient. Lubricant may alternatively be packaged in a foil or polymer packet which may also be packaged within the tray. Catheter 504 and/or drainage line 506 may be coiled so that they fit comfortably into the packaging tray. The tray may be designed to accommodate a single syringe only, or multiple syringes.

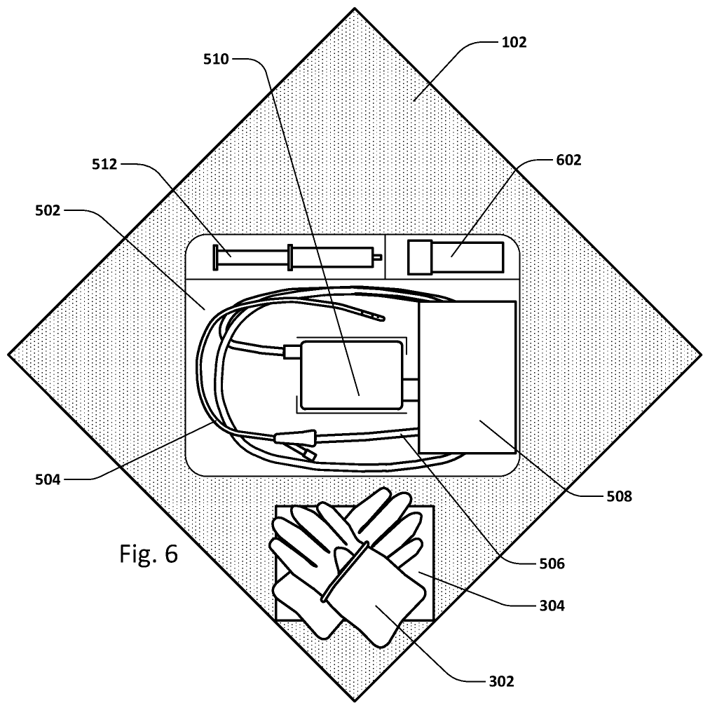

FIG. 6 shows the tray shown in FIG. 5 after top level tray 202 has been removed. Specimen bottle 602 may also be included in the tray.

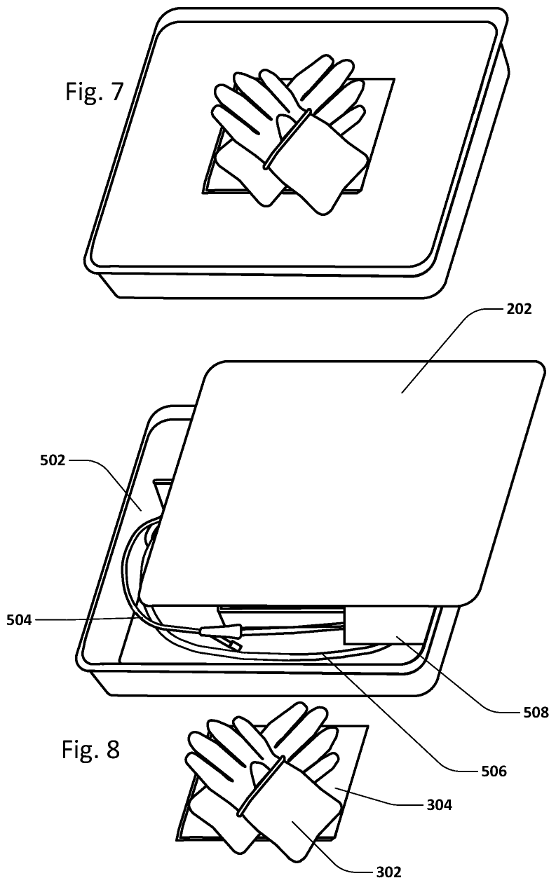

FIGS. 7 and 8 show an angled view of the tray shown in the above figures.

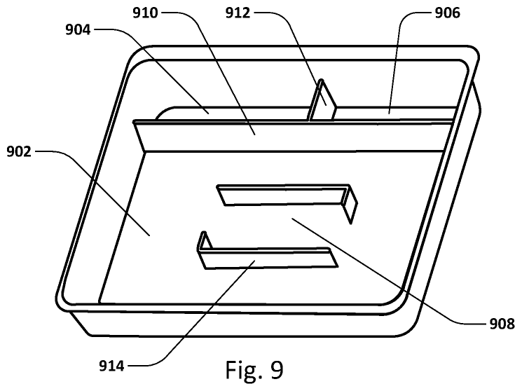

FIG. 9 shows lower tray 502 without its contents. Lower tray 502 includes main compartment 902, syringe compartment 904, sample bottle compartment 906, receptacle compartment 908. As well as main barrier 910, syringe barrier 912 and receptacle barriers 914. The barriers may be at the same height as each other, or at different heights. The barrier heights may be lower than the height of the outside wall of the lower tray. In this example, receptacle barriers 914 are lower than syringe barrier 912 and main barrier 910, which are lower than the outside wall of the lower tray. The barriers generally prevent contents of the tray from moving around excessively. FIG. 6 shows an example of which components may be packaged in which compartments. Syringe compartment 904 may have a relatively flat or uninclined base.

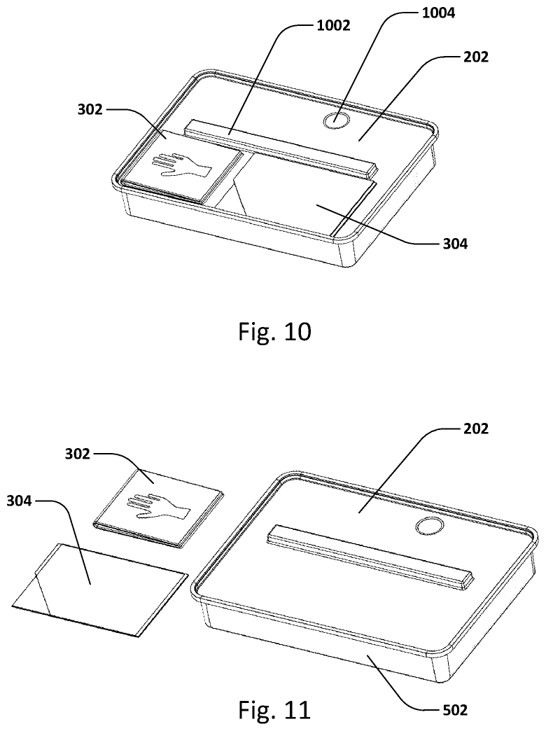

FIGS. 10 and 11 show another embodiment of the tray packaging system. Upper tray level 202 is shown here with ridge 1002 and opening 1004. Ridge 1002 serves to prevent the items on the upper tray from moving around excessively and/or provides rigidity to the upper tray and/or provides a “handle” to help remove the upper tray. One or more ridges may be present. For example, one horizontal ridge is shown here, but a horizontal ridge may be combined with one, two or more vertical ridges. The ridges may intersect each other. In this example, ridge 1002 is placed such that gloves 302 and drape 304 fit on one side of the ridge so that they do not slide around excessively. In some embodiments, more ridges may exist, for example, between gloves 302 and drape 304, or the ridges may define more precisely sized and shaped compartments designed specifically for different items. Opening 1004 may be used to grasp and lift upper tray level 202 to remove from lower tray 502, to access the items in the lower tray.

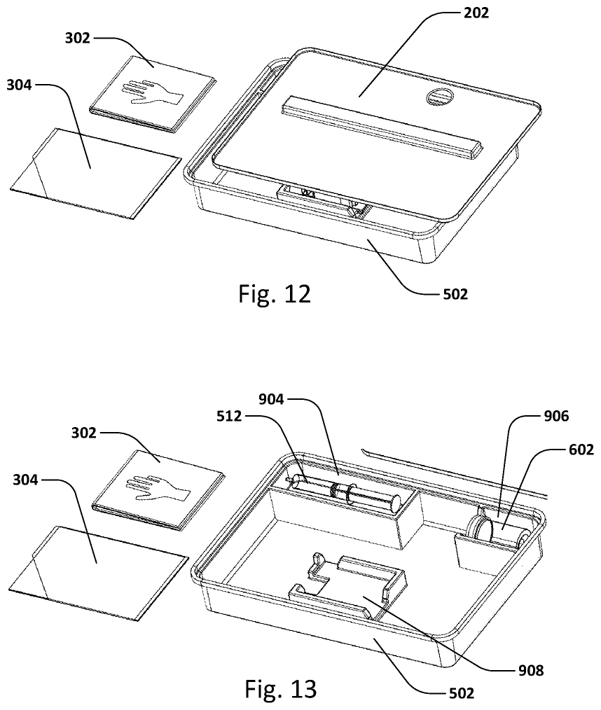

FIG. 12 shows the embodiment shown in FIGS. 10 and 11 as upper tray level 202 is being removed. FIG. 13 shows some of the contents of the lower tray in their compartments. Not shown here are catheter 504, drainage tube 506, receptacle 510 and fluid drainage bag 508. In some embodiments, lower tray level 502 is designed to accommodate only a single syringe, as shown here. In some embodiments, lower tray level 502 is designed to accommodate more than a single syringe.

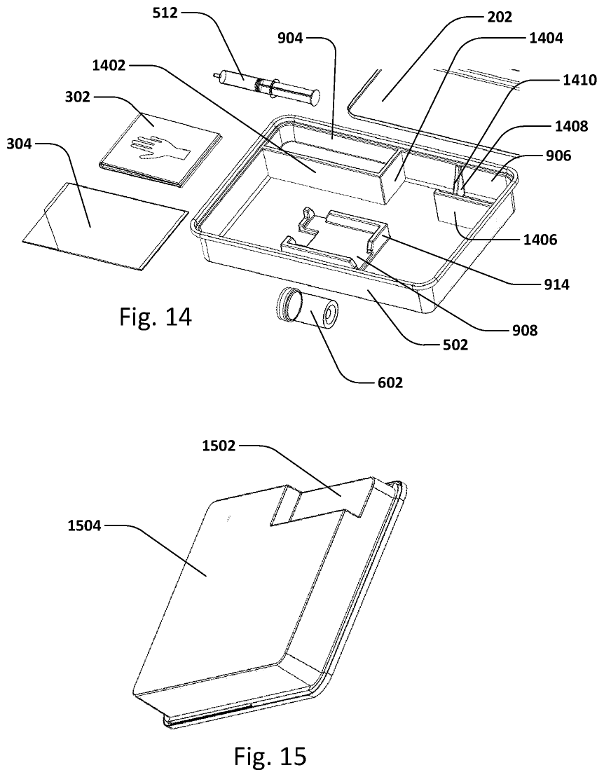

FIG. 14 shows the current embodiment with some of the contents removed to reveal the various compartments and barriers. This embodiment includes syringe compartment barriers or sidewalls 1402 and 1404 and specimen bottle compartment barriers 1406 and 1408. Barrier 1408 is shown here with lowered area 1410. Shown here are syringe compartment barriers which have a continuous ledge at a constant level with respect to the tray bottom. In other words, there is no lowered section to the syringe compartment barrier walls. Any of the barriers may or may not have a lowered section. It is understood that other items may be in these compartments and that fewer or more compartments may exist.

FIG. 15 shows the bottom of tray 502 shown in FIGS. 10-14. Note that the bottom of syringe compartment 904 is raised, which is illustrated by indentation 1502 in the bottom view of FIG. 15. The depth of the syringe compartment, or any compartment, may be reduced, or increased, based on the level of the bottom of the compartment. In this example, the depth of syringe compartment 904 is reduced. A shallower syringe compartment may serve as a reservoir for lubricant so that the catheter may be passed through the lubricant prior to insertion. A shallow compartment may aid in this purpose, although it is not necessary. A shallower compartment may avoid the need for a lowered area in the compartment barrier. Indentation 1502 may be relatively flat or uninclined, i.e. parallel to bottom 1504 of the tray.

Other items that may be included in the disclosed packaging include instructions for use, additional syringes, bottles, wraps, drapes, instructions, fluids, gloves, other medical devices, other catheters, etc.

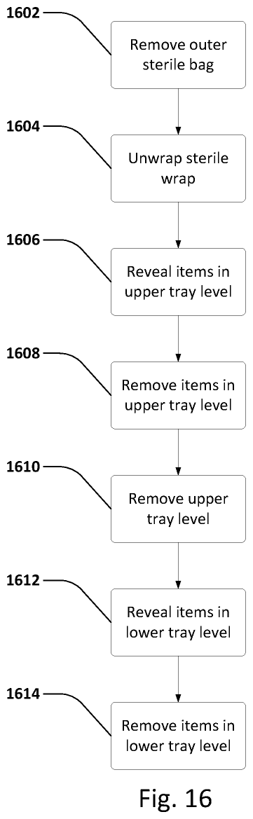

FIG. 16 shows some of the steps that may be used when using the disclosed packaging tray system. Step 1602 represents removing the tray package from any outer sterile bag or pouch. This pouch may be present to protect the packaging during transport etc., and to maintain the sterility of the packaging. The tray packaging may be sterilized in the sterile bag.

Step 1604 includes unwrapping the sterile wrap around the tray system. This serves both to lay a sterile area, defined by the unwrapped sterile wrap, and also to reveal, and make accessible, the contents of the top later of the tray. The outer sterile wrap is generally discarded since its outside surface is not sterile.

Step 1606 represents the revealing for access the contents of the upper tray level as the outer sterile wrap is unwrapped.

Step 1608 represents the removal of one or more items from the upper level of the tray. These items may be placed on the unwrapped sterile wrap, or they may be utilized, for example, gloves may be donned at this point. They may also be placed elsewhere, for example, instructions for use may be handed to an assistant or other person. The upper level tray may remain in the sterile field, or the lower tray may be discarded.

Step 1610 represents the removal of the upper tray level from a lower tray level, to reveal and access items in the lower tray level, as represented by step 1612.

Step 1614 represents accessing and/or removing some or all of the items in the lower tray level. The lower level tray may remain in the sterile field for use as a lubricant reservoir, or to house the procedure items until they are needed. Or the lower tray may be discarded.

In some embodiments the sterile wrap and/or sterile drape and/or other contents of the packaging may include instructing printed thereupon. In some embodiments, instructions may be all, or partly, in simple pictorial or graphic form. In some embodiments, the instructions may be placed so that they are apparent at the appropriate time during the procedure. For example, instructions for catheter prep and/or insertion, may be printed on the sterile drape which is draped over the legs and/or torso of the patient, so that the instructions are visible during the procedure.

Although a 2 level tray system is disclosed herein, it is understood that one, or more than 2 tray levels may be used.

Although generally rectangular trays are disclosed herein, it is understood that trays, and tray compartments, may take the form of alternative shapes, including polygons, circles, ovals, custom shapes, etc.

From the foregoing, it will be appreciated that specific embodiments of the invention have been described herein for purposes of illustration, but that various modifications may be made without deviating from the spirit and scope of the various embodiments of the invention. For example, several embodiments may include various suitable combinations of components, devices and/or systems from any of the embodiments described herein. Further, while various advantages associated with certain embodiments of the invention have been described above in the context of those embodiments, other embodiments may also exhibit such advantages, and not all embodiments need necessarily exhibit such advantages to fall within the scope of the invention.

What is claimed is:

1. A multi-level packaging assembly, comprising:
a top tray level;
a lower tray level upon which the top tray level is removably positioned;
one or more compartment barriers which define a first compartment within the lower tray level such that the first compartment is sized to accommodate a single syringe, wherein the first compartment barriers have a continuous ledge at a constant level with respect to a bottom of the lower tray level;
a base defined within the first compartment, wherein the base is uninclined relative to the bottom of the lower tray level; and
at least one layer of wrap sized so as to fold over and enclose both the lower tray level and the top tray level when the top tray level is positioned upon the lower tray level such that unfolding of the at least one layer of wrap reveals the top tray level.

2. The assembly of claim 1 where folds or corners of the at least one layer of wrap are free of any items.

3. The assembly of claim 2 wherein folds or corners of the at least one layer of wrap are free of hand sanitizer, additional wrap, and instructions.

4. The assembly of claim 1 wherein the top tray level comprises at least one ridge along a surface of the top tray level.

5. The assembly of claim 1 further comprising at least one pair of gloves positioned upon the top tray level.

6. The assembly of claim 1 further comprising at least one drape in a folded configuration positioned upon the top tray level.

7. The assembly of claim 1 further comprising a single syringe positioned within the first compartment.

8. The assembly of claim 1 wherein the lower tray level further defines a second compartment adjacent to the first compartment.

9. The assembly of claim 8 wherein the second compartment is sized for receiving a sample bottle.

10. The assembly of claim 8 wherein the lower tray level further defines a third compartment having one or more additional compartment barriers defined within the third compartment.

11. The assembly of claim 10 further comprising one or more instruments positioned within the third compartment.

12. The assembly of claim 11 wherein the one or more instruments comprise a catheter, a drainage line, a fluid receptacle, a drainage bag, or combinations thereof.

13. A method of using a multi-level packaging assembly, comprising:
unfolding at least one layer of wrap enclosing a top tray level positioned upon a lower tray level to reveal the top tray level;
removing the top tray level from the lower tray level to access the lower tray level,
wherein the lower tray level comprises one or more compartment barriers having a continuous ledge at a constant level with respect to a bottom of the lower tray level and which define a first compartment having a single syringe positioned within the first compartment, and
wherein the first compartment further defines a base which is uninclined relative to the bottom of the lower tray level.

14. The method of claim 13 wherein unfolding at least one layer of wrap comprises revealing at least one pair of gloves positioned upon the top tray level.

15. The method of claim 13 wherein unfolding at least one layer of wrap comprises revealing at least one drape in a folded configuration positioned upon the top tray level.

16. The method of claim 13 further comprising accessing a single syringe positioned within the first compartment.

17. The method of claim 13 further comprising accessing one or more instruments positioned within an additional compartment defined within the lower tray level.

18. The method of claim 17 wherein accessing one or more instruments further comprises accessing a catheter, a drainage line, a fluid receptacle, a drainage bag, or combinations thereof from within the lower tray level.

19. The method of claim 17 wherein unfolding at least one layer of wrap comprises unfolding folds or corners of the at least one layer of wrap which are free of any items.

20. The method of claim 19 wherein unfolding at least one layer of wrap comprises unfolding folds or corners of the at least one layer of wrap which are free of hand sanitizer, additional wrap, and instructions.

21. The method of claim 17 wherein the top tray level comprises at least one ridge along a surface of the top tray level.

22. A method of producing a multi-level packaging assembly, comprising:
providing a lower tray level having one or more compartment barriers and a base which together define a first compartment within the lower tray level, wherein the one or more compartment barriers have a continuous ledge at a constant level with respect to a bottom of the lower tray level, and wherein the base is uninclined relative to the bottom of the lower tray level;
securing a single syringe within the first compartment and one or more instruments within the lower tray level;
positioning a removable top tray level upon the lower tray level; and
enclosing the lower tray level and the top tray level with at least one layer of wrap such that unfolding of the at least one layer of wrap reveals the top tray level.

23. The method of claim 22 wherein securing one or more instruments within the lower tray level comprises positioning a catheter, a drainage line, a fluid receptacle, a drainage bag, or combinations thereof.

24. The method of claim 22 wherein positioning the removable top tray level further comprises positioning at least one pair of gloves upon the top tray level prior to enclosing the lower tray level and top tray level.

25. The method of claim 22 wherein positioning the removable top tray level further comprises positioning at least one drape in a folded configuration positioned upon the top tray level prior to enclosing the lower tray level and top tray level.

26. The method of claim 22 where folds or corners of the at least one layer of wrap are free of any items.

27. The method of claim 26 wherein folds or corners of the at least one layer of wrap are free of hand sanitizer, additional wrap, and instructions.

28. The method of claim 22 wherein the top tray level comprises at least one ridge along a surface of the top tray level.
